(19) United States
(12) Patent Application Publication
(10) Pub. No.: US 2017/0251973 A1
(43) Pub. Date: Sep. 7, 2017

(54) Process for Measuring Tumor Response to an Initial Oncology Treatment

(71) Applicant: Razelle Kurzrock, San Diego, CA (US)

(72) Inventor: Razelle Kurzrock, San Diego, CA (US)

(73) Assignee: Novena Therapeutics Inc., San Diego, CA (US)

(21) Appl. No.: 15/057,965

(22) Filed: Mar. 1, 2016

(51) Int. Cl. A61B 5/00; C12Q 1/68; A61B 6/03

(52) U.S. Cl. CPC A61B 5/4848; A61B 6/037; A61B 5/4839; C12Q 1/6886; C12Q 2600/106; C12Q 2600/156

(57) ABSTRACT

There is disclosed a process for determining early patient response to an initial therapy administration, comprising a rapid determination of effectiveness of a therapy after an initial treatment for a cancer indication. More specifically, the rapid determination of effectiveness is made days following initial therapy. There is further disclosed a process for determining early patient response to an initial cancer treatment, comprising (a) conducting a baseline PET FDG or PET FLT scan of the tumor region in a patient by determining tumor tissue metabolic rate and/or apoptotic rate (for FLT), (b) providing a single potentially effective dose of a therapeutic to the patient, (c) conducting a second PET FDG or PET FLT scan by determining tumor tissue metabolic rate, and (d) comparing the results of the first PET scan to the second PET scan to determine an imaging response in the PET scan results whereby at least a 1% reduction in tumor tissue metabolic rate indicates that the patient would benefit to treat the tumor with the initial cancer treatment. Preferably, in addition to the PET scans conducted before and after an initial dose of a cancer drug, the present disclosure further provides parallel before and after measurement(s) of the level of circulating tumor DNA (ctDNA) for specific oncogene mutations/alterations to identify early patient response to drug therapy.

### CROSS REFERENCE TO RELATED APPLICATION

This patent application claims priority from U.S. provisional patent application 62/126,682 filed 1, Mar. 2015.

### TECHNICAL FIELD

The present disclosure provides a process for determining early patient response to an initial therapy administration, comprising a rapid determination of an effectiveness of a therapy after an initial treatment for a cancer indication. More specifically, the rapid determination of effectiveness is made within one to fourteen days following initial therapy. The present disclosure further provides a process for determining early patient response to an initial cancer treatment, comprising (a) conducting a baseline PET FDG or PET FLT scan of the tumor region in a patient by determining tumor tissue metabolic rate and/or tumor apoptosis (for FLT) (b) providing a single potentially effective dose of a therapeutic to the patient, (c) conducting a second PET FUG or PET FLT scan to further determine tumor tissue metabolic rate and/or apoptosis, and (d) comparing the results of the first PET scan to the second PET scan to determine an imaging response in the PET scan results whereby at least a 1% reduction in tumor tissue metabolic rate and/or apoptosis (latter for FLT) indicates that the patient would benefit from the treatment. Preferably, in addition to the PET scans conducted before and after an initial dose of a cancer drug, the present disclosure further provides a parallel before and after measurement of the level of circulating tumor DNA (ctDNA) (found in blood or urine or other body fluids) for specific oncogene mutations/alterations to identify early patient response to drug therapy.

### BACKGROUND

The standard to monitor therapy response in solid tumors has been measurements of tumor size by CT (CAT scan) or MRI, using RECIST (Response Evaluation Criteria in Solid Tumors) criteria for solid tumors, WHO criteria for lymphomas, and similar criteria for other subsets of disease. A limitation of this method is that changes in tumor size cannot be accurately assessed for many weeks and sometimes months after the initiation of tumor treatment, and therefore the patient must continue on treatment even though the patient may not be benefitting or may even be getting worse and the patient has to tolerate side effects of the treatment. The opposite can occur as well. Tumors may be improving but show an initial increase in size when measured by CAT scan or MRI or other imaging. This can occur because of many reasons including inflammatory changes. Therefore, a patient who is getting worse may stay on an ineffective treatment for months, and a patient whose tumor is getting better may have the treatment stopped.

Often, scans that measure tumor size are not repeated for 6 to 12 weeks after treatment starts because earlier scans do not reveal measurable changes. Therefore, it is routine that patients whose tumors are actually getting worse (that is, disease progression) stay on ineffective treatments for at least 6 to 12 weeks and often longer. The opposite is also true. In some patients, the treatment, as intended, causes beneficial cell death and necrosis or interferes with tumor metabolism but does not cause immediate shrinkage in tumor size. Indeed, in some cases, there can be initial growth of tumor (perhaps due to inflammation or other changes) even though a beneficial response is occurring (see Kurzrock et al Ann. Oncol. 2013 September; 24(9):2256-61. http://www.ncbi.nlm nih.gov/pubmed/23676418 and Benjamin et al. Clin. Oncol. 2007 May 1; 25(13):1760-4. http://www.ncbi.nlm nih.gov/pubmed/17470866). The latter is a well-described phenomenon in several forms of neoplasms and especially when new targeted or immunotherapy treatments are used. In these patients, the imaging repeated at 6 to 12 weeks after treatment start may indicate tumor growth and the patients may have an effective treatment stopped.

Moreover, there is also a need in the art to better segment patients for clinical trials because often highly effective therapies are missed when the patients are not better segmented for responders so as to identify patient populations who would benefit from a drug from those populations of patients who do not respond to such a drug. For example, several recombinant antibodies to insulin-like growth factor 1 (IGF-1R) showed only modest activity in larger scale clinical trials in patients with relapsed or refractory Ewing sarcoma family of tumors (ESFT). But closer observations showed a subgroup of patients who showed very significant beneficial responses to this relatively safe tumor therapy. (Chen and Sharon “IGF-1R as an anti-cancer target-trials and tribulations” Chin. J. Cancer, 2013 May; 32(5):242-52.). Therefore, there is a need in the art to identify the subgroups of patients who would benefit from a targeted cancer treatment (such as, anti-IGF-1R antibody treatment) such that subgroup effective treatments can be identified and subsequently approved with proper patient segmentation. The present disclosure was made in an effort to identify early such subgroups.

[¹⁸F] fluorodeoxyglucose (¹⁸F-FDG) uptake has been measured before and after induction chemotherapy in pediatric patients with ESFT (Ewings sarcoma) and response correlated with assessment of other types of imaging and with outcome (Hawkins et al., J. Clin. Oncol. 23:8828-8834, 2005). In another group of patients with osteosarcoma treated with chemotherapy ¹⁸F-FDG positron emission tomography (PET) correlated with histological response (Im et al., Eur. J. Nucl. Med Mol. Imaging 39:39-49, 2012). But these studies used PET imaging at traditional times, that is, after 6 weeks or months of chemotherapy.

A direct mode of action of cancer therapeutic agents often translates into meaningful measurable effects (such as, tumor shrinkage in baseline (index) lesions) after a few weeks or months of initial administration. Studies have indicated that achieving a response after the initial cycles of chemotherapy is predictive of complete remission (CR) and improved survival (von Minckwitz et al. Breast Cancer Res. 2008; 10:R30; and Hutchings et al. Blood 2006, 107:52-59.). Response criteria for solid tumors or lymphomas were developed by the WHO in an attempt to standardize the characterization of therapeutic efficacy and to facilitate comparisons between studies as well as comparisons with historical data (WHO handbook for reporting results of cancer treatment. Geneva (Switzerland): World Health Organization Offset Publication No. 48, 1979; and Miller et al., Cancer 1981, 47:207-214). Simplified and standardized response definitions for solid tumors were published by the RECIST Group in 2000 (Therasse et al., J. Natl. Cancer Inst. 2000, 92:205-216.). For cytotoxic agents, these guidelines assumed that an increase in tumor growth and/or the appearance of new lesions, usually assessed by CT scans or MRIs about eight weeks after starting the drug, and then every 8 to 12 weeks thereafter, signaled progressive disease (PD), such that the term “progression” became synonymous with drug failure. Cessation of the current treatment is recommended once PD is detected.

Increasing clinical experience indicates that traditional response criteria may not be sufficient to fully characterize activity in this new era of targeted therapies and/or biologics. For example, stable disease (SD) is characterized as either an increase or a decrease in tumor burden insufficient in magnitude to qualify as PD or a partial response (PR), respectively. With chemotherapy, SD is often transient and therefore not considered indicative of true antitumor activity. In contrast, with tyrosine kinase inhibitors (e.g., targeting epidermal growth factor receptor in non-small cell lung cancer), achieving SD has been identified as a potential surrogate end point for improved clinical outcome (median time to progression; Tsujino et al. J. Clin. Oncol. 2008; 26). Interpretation of this end point under the WHO and RECIST criteria, therefore, has been revisited in recent years, and durable modest regressions or prolonged SD achieved by these agents is, in some cases, now viewed as evidence of activity (Ratain and Eckhardt J. Clin. Oncol. 2004, 22:4442-4445).

Molecular markers are also used to better understand disease. However, these are classically utilized before treatment, and, if done later, they are repeated at the time of tumor progression on scans (many weeks or months after treatment). For instance, Lewis et al. (Modern Pathology 20:397-404, 2007) conducted a PCR test for translocation for Ewing's sarcoma for EWS-FLI1, EWS-ERG, EWS-ETV1, EWS-ETV4 and EWS-FEV as to identify appropriate translocation tumor markers. However, Lewis et al was not able to utilize the response biomarkers identified for this tumor type to be able to quickly monitor whether an initial dose of therapy can lead to tumor regression or tumor progression.

Therefore, there is a need in the art to rapidly measure the effectiveness of a cancer treatment drug or drug regimen (multiple drugs administered during a single course of therapy) in order to measure patient benefit quickly in view of the multitude of serious side effects that cancer treatment entails. This need is not only for the patient treatment situation but also for clinical trials of therapies wherein subgroups can be identified early in a treatment cycle to provide approvable criteria to determine which patients are appropriate for subgroup criteria that are within the label indications of an approved therapeutic or therapeutic combination.

RECIST defines response so RECIST is supposed to correlate with response by definition. But, there is controversy whether response correlates with survival. It may depend on the class of therapeutic, such as a lack of correlation with highly cytotoxic drugs because patient survival is compromised by toxicity. But in the situation of a targeted therapy, the correlation between a response and survival is high.

Accordingly, the main problem with current response criteria is that the patients must stay on drug for 8+ weeks even if their tumors are progressing. Assessment of response generally occurs at about 8 weeks by a scan, with reassessment about every 8 to 12 weeks thereafter. If we knew early (say on day 5) whether or not the patient was likely to respond, patients who were likely to be non-responders could move on to more effective treatments.

Even for drugs with few or no side effects, the need to be able to predict response is crucial. That is because, with traditional paradigms, response assessment is done after 8 weeks. For patients with cancer who are not responding, their tumor has been allowed to grow for those eight weeks. Current methods for prediction of response concentrate on assessment of pretreatment tumor specimens for biomarkers that might predict response to the targeted agent given. The appropriate biomarker to be used would of course vary by the agent given. And because tumors evolve and change, genuinely accurate prediction would require acquiring new tissue before each treatment, knowing the specific biomarker that is predictive for that treatment, and assaying for that biomarker. Biomarker prediction can be complicated because many patients with the predictive biomarker may still not respond, since tumors often have multiple defects, and some of the additional defects may create resistance pathways even in the presence of the predictive biomarker.

Further, the other problem with week 8 assessments of response relates to abandoned drugs (i.e., failed clinical trials). If the response rate is 15% and a tumor incidence is rare, a large randomized study is needed to prove efficacy to the FDA. But a randomized study may need to include thousands of patients to see a difference in outcome if response rates are low. The trial is likely to be negative unless very large numbers of patient are assessed because 85% of patients are, in effect, being harmed by the drug by having to take it for 8+ weeks before response is known. And once the drug is approved, if the trial can be done, most patients are not benefitting or are being harmed. Hence, there is a need for an accurate response predictor very early.

Accordingly, there is a need in the art to predict (non-pharmacokinetic) patient susceptibility to various kinase inhibitors, antibodies and other agents by actually administering a potentially effective dose rather than a radiolabeled micro-dose of the drug.

### SUMMARY

The present disclosure provides a process for determining early patient response to an initial therapy administration, comprising a rapid determination of an effectiveness of a therapy after an initial treatment for a cancer indication. More specifically, the rapid determination of effectiveness is made within 1-14 days following initial therapy. The present disclosure provides a process for determining early patient response to an initial cancer treatment, comprising (a) conducting a baseline PET FDG or PET FLT scan of the tumor region in a patient for determining tumor tissue metabolic rate and/or tissue apoptosis; (b) providing a single potentially effective dose of a targeted therapeutic to the patient; (c) conducting a second PET FDG or PET FLT scan by determining tumor tissue metabolic rate; and (d) comparing the results of the first PET scan to the second PET scan to determine an imaging response in the PET scan results whereby at least a 1% reduction in tumor tissue metabolic rate indicates that the patient would benefit to treat the tumor with the initial cancer treatment. Preferably, in addition to the PET scans conducted before and after an initial dose of a cancer drug, the present disclosure further provides a parallel before and after serial measurement of the level of circulating tumor DNA (ctDNA) (measured in blood, urine or other body fluids) for specific oncogene mutations/alterations based on a biomarker specific for a tumor to identify early patient response to drug therapy.

Preferably, the PET scans determine lesion volume VOI ROIs to obtain maximum SUVmax and SUV SUV minimum SUVmin. Preferably, the present disclosure further comprises an additional urine or blood test to quantitate a cell-free DNA aberration representing a cancer marker that is conducted in conjunction with each PET scan to confirm changes caused by a single therapeutic dose of a cancer therapeutic.

Preferably, the PET scans are conducted with 2-deosy-2 [¹⁸F] fluoro D-glucose (FDG) PET scans or [18F]-fluoro-3′-deoxy-3′-L: -fluorothymidine ([¹⁸F]FLT) PET scans. Preferably, the second PET scan is conducted within 14 days after completion of the first dose of the therapeutic to the patient. Preferably, the therapeutic is a targeted therapeutic, such as a kinase inhibitor or an antibody.

The present disclosure provides a process for determining patient subgroup inclusion in a clinical trial of a targeted therapy for cancer, comprising (a) conducting a baseline PET FDG or PET FLT scan of the tumor region in a patient by determining baseline tumor tissue metabolic rate, (b) providing a single dose of a clinical trial test targeted therapeutic to the patient, (c) conducting a second PET FUG or PET FLT scan within 1-14 days following the single dose, by determining tumor tissue metabolic rate, and (d) comparing the results of the first PET scan to the second PET scan to determine an imaging response in the PET scan results, whereby at least a 1% reduction in tumor tissue metabolic rate indicates that the patient would be included in the clinical trial subgroup. Preferably, the process further comprises conducting a DNA aberration test comprising (i) conducting a baseline measurement of the level of circulating tumor DNA (ctDNA) for a specific oncogene mutations/alterations from a response biomarker specific for a tumor to identify early patient response to drug therapy, wherein the response biomarker is selected from the group consisting of the response biomarkers in Tables 1A and 1B, (ii) providing a single potentially effective dose of a therapeutic to the patient, (iii) conducting a second or serial measurement(s) of the level of circulating tumor DNA (ctDNA) for specific oncogene mutations/alterations from the response biomarker specific for a tumor to identify early patient response to drug therapy, wherein the second or serial measurements are conducted 1-14 days following the dose of the therapeutic, and (iv) comparing the results of the first measurement to subsequent measurements of ctDNA to determine that early reduction in ctDNA of at least 1% indicates that the patient would benefit to continue to treat the tumor with the therapeutic.

Preferably the response biomarker is selected from the group consisting of ABL1, BRAF, CHEK1, FANCC, GATA3, JAK2, MITF, PDCD1LG2, RBM10, STAT4, ABL2, BRCA1, CHEK2, FANCD2, GATA4, JAK3, MLH1, PDGFRA, RET. STK11, ACVR1B, BRCA2, CIC, FANCE, GATA6, JUN, MPL, PDGFRB, RICTOR, SUFU, AKT1, BRD4, CREBBP, FANCF, GID4 (C17or 39), KAT6A (MYST3), MRE11A, PDK1, RNF43, SYK, AKT2, BRIP1, CRKL, FANCG, GLI1, KDM5A, MSH2, PIK3C2B, ROS1, TAF1, AKT3, BTG1, CRLF2, FANCL, GNA11, KDM5C, MSH6, PIK3CA, RPTOR, TBX3, ALK, BTK, CSF1R, FAS, GNA13, KDM6A, MTOR, PIK3CB, RUNX1, TERC, AMER1 (FAM123B), C11 or 30 (EMSY), CTCF, FAT1, GNAQ, KDR, MUTYH, PIK3CG, RUNX1T1, TERT (promoter only), APC, CARD11, CTNNA1, FBXW7, GNAS, KEAP1, MYC, PIK3R1, SDHA, TET2, AR, CBFB, CTNNB1, FGF10, GPR124, KEL, MYCL (MYCL1), PIK3R2, SDHB, TGFBR2, ARAF, CBL, CUL3, FGF14, GRIN2A, KIT, MYCN, PLCG2, SDHC, TNFAIP3, ARFRP1, CCND1, CYLD, FGF19, GRM3, KLHL6, MYD88, PMS2, SDHD, TNFRSF14, ARID1A, CCND2, DAXX, FGF23, GSK3B, KMT2A (MLL), NF1, POLD1, SETD2, TOP1, ARID1B, CCND3, DDR2, FGF3, H3F3A, KMT2C (MLL3), NF2 POLE, SF3B1, TOP2A, ARID2, CCNE1, DICER1, FGF4, HGF, KMT2D (MLL2), NFE2L2, PPP2R1A, SLIT2, TP53, ASXL1, CD274, DNMT3A, FGF6, HNF1A, KRAS, NFKBIA, PRDM1, SMAD2, TSC1, ATM, CD79A, DOT1L, FGFR1, HRAS, LMO1, NKX2-1, PREX2, SMAD3, TSC2, ATR, CD79B, EGFR, FGFR2, HSD3B1, LRP1B, NOTCH1, PRKAR1A, SMAD4, TSHR, ATRX, CDC73, EP300, FGFR3, HSP9OAA1, LYN, NOTCH2, PRKCI, SMARCA4, U2AF1, AURKA, CDH1, EPHA3, FGFR4, IDH1, LZTR1, NOTCH3, PRKDC, SMARCB1, VEGFA, AURKB, CDK12, EPHA5, FH, IDH2, MAGI2, NPM1, PRSS8, SMO, VHL, AXIN1, CDK4, EPHA7, FLCN, IGF1R, MAP2K1, NRAS, PTCH1, SNCAIP, WISP3, AXL, CDK6, EPHB1, FLT1, IGF2, MAP2K2, NSD1, PTEN, SOCS1, WT1, BAP1, CDK8, ERBB2, FLT3, IKBKE, MAP2K4, NTRK1, PTPN11, SOX10, XPO1, BARD1, CDKN1A, ERBB3, FLT4, IKZF1, MAP3K1, NTRK2, QKI, SOX2, ZBTB2, BCL2, CDKN1B, ERBB4, FOXL2, IL7R, MCL1, NTRK3, RAC1, SOX9, ZNF217, BCL2L1, CDKN2A, ERG, FOXP1, INHBA, MDM2, NUP93, RAD50, SPEN, ZNF703, BCL2L2, CDKN2B, ERRFIL FRS2, INPP4B, MDM4, PAK3, RAD51, SPOP, BCL6, CDKN2C, ESR1, FUBP1, IRF2, MED12, PALB2, RAF1, SPTA1, BCOR, CEBPA, EZH2, GABRA6, IRF4, MEF2B, PARK2, RANBP2, SRC, BCORL1, CHD2, FAM46C, GATA1, IRS2, MEN1, PAX5, RARA, STAG2, BLM, CHD4, FANCA, GATA2, JAK1, MET, PBRM1, RB1, STATS, and combinations thereof.

Preferably the DNA aberration biomarker test that is conducted at substantially the same time as the first PET scan and the second DNA aberration biomarker test is conducted at the same time as the second PET scan. Preferably, select DNA rearrangements are also response biomarkers and are selected from the group consisting of the genes ALK, BRAF, EPOR, ETV6, IGK, JAK2, NTRK1, RAF1, ROS1, BCL2, CCND1, ETV1, EWSR1, IGL, KMT2A (MLL), PDGFRA, RARA, TMPRSS2, BCL6, CRLF2, ETV4, FGFR2, JAK1, MYC, PDGFRB, RET, TRG, BCR, EGFR, ETVS, IGH, and combinations thereof. Further, select gene fusion genes are selected from the group consisting of ABI1, CBFA2T3, EIF4A2, FUS, JAK1, MUC1, PBX1, RNF213, TET1, ABL1, CBFB, ELF4, GAS7, JAK2, MYB, PCM1, ROS1, TFE3, ABL2, CBL, ELL, GLI1, JAK3, MYC, PCSK7, RPL22, TFG, ACSL6, CCND1, ELN, GMPS, JAZF1, MYH11, PDCD1LG2 (PDL2), RPN1, TFPT, AFF1, CCND2, EML4, GPHN, KAT6A (MYST3), MYH9, PDE4DIP, RUNX1, TFRC, AFF4, CCND3, EP300, HERPUD1, KDSR, NACA, PDGFB, RUNX1T1 (ETO), TLX1, ALK, CD274 (PDL1), EPOR, HEY1, KIFSB, NBEAP1 (BCL8), PDGFRA, RUNX2, TLX3, ARHGAP26 (GRAF), CDK6, EPS15, HIP1, KMT2A (MLL), NCOA2, PDGFRB, SEC31A, TMPRSS2, ARHGEF12, CDX2, ERBB2, HIST1H4I, LASP1, NDRG1, PERI, SEPTS, TNFRSF11A, ARID1A, CHIC2, ERG, HLF, LCP1, NF1, PHF1, SEPT6, TOP1, ARNT, CHN1, ETS1, HMGA1, LMO1, NF2, PICALM, SEPT9, TP63, ASXL1, CIC, ETV1, HMGA2, LMO2, NFKB2, PIM1, SET, TPM3, ATF1, CIITA, ETV4, HOXA11, LPP, NIN, PLAG1, SH3GL1, TPM4, ATG 5, CLP1, ETV5, HOXA13, LY L 1, NOTCH1, PML, SLC1A2, TRIM24, ATIC, C LTC, ETV6, HOXA3, MAF, NPM1, POU2AF1, SNX29 (RUND-C2A), TRIP11, BCL10, CLTCL1, EWSR1, HOXA9, MAFB, NR4A3, PPP1CB, SRSF3, TTL, BCL11A, CNTRL (CEP110), FCGR2B, HOXC11, MALT1, NSD1, PRDM1, SS18, TYK2, BCL11B, COL1A1, FCRL4, HOXC13, MDS2, NTRK1, PRDM16, SSX1, USP6, BCL2, CREB3L1, FEV, HOXD11, MECOM, NTRK2, PRRX1, SSX2, WHSC1 (MMSET or NSD2), BCL3, CREB3L2, FGFR1, HOXD13, MKL1, NTRK3, PSIP1, SSX4, WHSC1L1, BCL6, CREBBP, FGFR1OP, HSP90AA1, MLF1, NUMA1, PTCH1, STAT6, YPELS, BCL7A, CRLF2, FGFR2, HSP90AB1, MLLT1 (ENL), NUP214, PTK7, STL, ZBTB16, BCL9, CSF1, FGFR3, IGH, MLLT10 (AF10), NUP98, RABEP1, SYK, ZMYM2, BCOR, CTNNB1, FLI1, IGK, MLLT3, NUTM2A, RAF1, TAF15, ZNF384, BCR, DDIT3, FNBP1, IGL, MLLT4 (AF6), OMD, RALGDS, TALI, ZNF521, BIRC3, DDX10, FOXO1, IKZF1, MLLT6, P2RY8, RAP1GDS1, TAL2, BRAF, DDX6, FOXO3, IL21R, MN1, PAFAH1B2, RARA, TBL1XR1, BTG1, DEK, FOXO4, IL3, MNX1, PAX 3, RBM15, TCF3 (E2A), C AMTA 1, DUSP22, FOXP1, IRF4, MSI2, PAX 5, RET, TCL1A (TCL1), CARS, EGFR, FSTL3, ITK, MSN, PAX 7, RHOH, TEC, and combinations thereof.

The present disclosure further provides a process for determining whether a patient would benefit for cancer treatment with a particular therapeutic, comprising (a) conducting a baseline measurement of the level of circulating tumor DNA (ctDNA) for specific oncogene mutations/alterations from a response biomarker specific for a tumor to identify early patient response to drug therapy, wherein the response biomarker is selected from the group consisting of the response biomarkers in Tables 1A and 1B, (b) providing a single potentially effective dose of a therapeutic to the patient, (c) conducting a second or serial measurement(s) of the level of circulating tumor DNA (ctDNA) for specific oncogene mutations/alterations from a response biomarker specific for a tumor, wherein the response biomarker is selected from the group consisting of the response biomarkers in Tables 1A and 1B, wherein the second or serial measurement(s) are conducted 1-14 days following the dose of the therapeutic, and (d) comparing the results of the first measurement to subsequent measurements of ctDNA to determine that early reduction in ctDNA of at least 1% indicates that the patient would benefit to continue to treat the tumor with the therapeutic. Preferably, the second or serial measurement(s) is conducted from 1-10 days after completion of a first dose of a therapeutic to the patient.

### BRIEF DESCRIPTION OF THE FIGURE

FIG. 1 shows at PET scan of a person's tumor conducted prior to treatment (left) and two days after treatment (right) demonstrating early metabolic response.

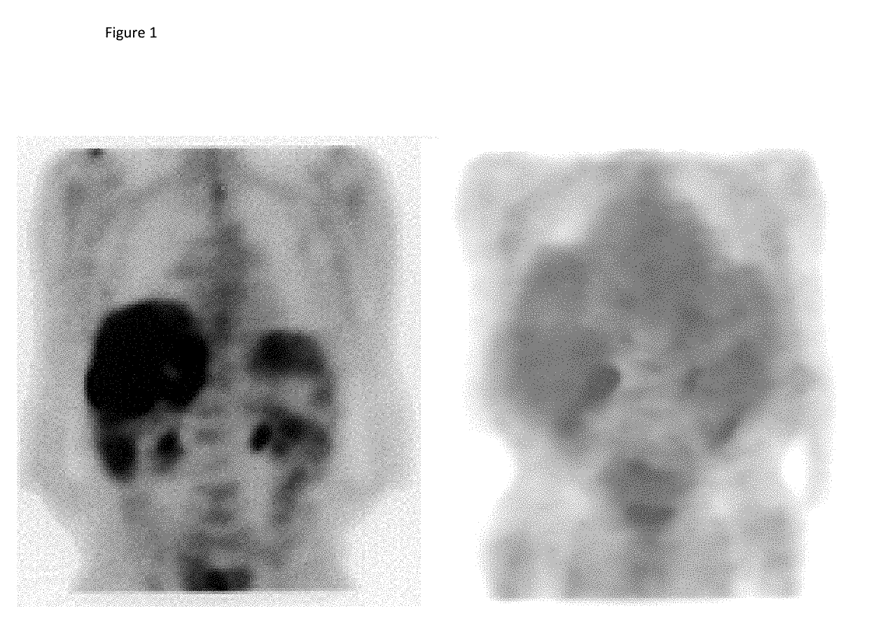

### DETAILED DESCRIPTION

The present disclosure provides using PET imaging in the first 14 days after treatment (and compare to pretreatment PET imaging) to be able to predict which patients will respond and who will not. This is applicable to patients with Ewing sarcoma or in general to patients with cancer treated with a variety of modalities. The ability of ¹⁸F-FDG PET imaging to be an early indicator of response and predict survival has been recently corroborated in Ewing sarcoma (Joo Hyun O¹, et al. Response to early treatment evaluated with ¹⁸F-18F-FDG PET and PERCIST 1.0 predicts survival in patients with Ewing sarcoma family of tumors treated with a monoclonal antibody to the insulin-like growth factor 1 receptor. Journal of Nuclear Medicine, published on Jan. 21, 2016 as doi:10.2967/jnumed.115.162412) which is not prior art to the priority provisional patent application filed on 1 Mar. 2015.

An early marker of response, as provided herein solves several problems: (i) it permits patients who are not benefitting from therapy to be taken off that therapy and moved to another treatment after just a few days (or at most 14 days), rather than 6 weeks or months; (ii) it allows better assessment of who might be benefitting from therapy and reduces the chances that a patient with true benefit might be removed from treatment because CAT or MRI or similar imaging measuring tumor size shows “pseudogrowth” even while the tumor is dying; and (iii) it permits the early selection of responders for clinical trials such that a greater percentage of drugs will achieve statistical significance for efficacy and subsequent commercial approval with labeling requiring/suggesting that the presently disclosed procedure be conducted after a single treatment dose in order to determine who stays on the treatment and who gets moved to a different treatment. This is important because drugs that produce definitive responses, but in only small subsets of patients, may not be approvable in unselected patient populations. Yet these drugs can be successful once there is the ability to select the subgroup of responders within days of the first dose of drug.

The present disclosure provides a method for determining the effectiveness of a kinase inhibitor drug with a patient using a PET image, comprising the steps of: (1) obtaining three-dimensional images of different PET images at the time of or just prior to kinase drug administration of a therapeutic dose; (2) administering a single therapeutic dose to the, patient and (2) determining the lesion volume VOI ROIs to obtain maximum SUV/max and SUV SUV/minimum SUVmin; (3) in the same direction to cut the three-dimensional PET images obtained several faults, and each fault in the corresponding region of interest in the lesion volume VOI The lesion outline the region of interest based on a number of SUV and other activity values on the line ROI; (4) the three-dimensional PET image following the same line of the corresponding volume of the SUV activity as the reference value, the activity of drawing lines of different volumes and the corresponding Activity—volume curve; (5) calculating the time of administration of the specific activity—the area under the line of the curve of the volume; (6) in accordance with PET images acquired under different administration times to obtain and calculate different administration activity of time under the volume relationship line area under the curve, the effectiveness of drug is determined using T-test for statistical analysis.

The presently disclosed method uses early response to predict response. It is applicable to a wide range of tumors and agents, and would not require understanding the precise role of the biomarker portfolio in the tumor. Further, the use of early response biomarkers to predict tumor response, even if the overall response rate is 15% or even less, can find those patients who will be responders by giving them only one dose of drug. Therefore, in the context of oncology clinical drug trial designs, the present disclosed method to find responders 3-7 days following a single drug administration, significantly improves probability of running a successful clinical drug trial and improves overall drug approval rates.

As many as 85% of patients are harmed by a drug treatment, due to staying on an ineffective drug for eight weeks or longer before anyone realizes the drug is not effective for the patient's tumor, and having alternative perhaps more effective therapy delayed. The present method is able to determine treatment effectiveness at a much earlier time in order to change ineffective treatments earlier or to determine which patients should continue on a clinical study. For example, if 50% of the patients that are predicted to respond by the present early predictive method do respond (Joo Hyun O¹ et al. Journal of Nuclear Medicine, published on Jan. 21, 2016 as doi:10.2967/jnumed.115.162412), and the overall response rate in an unselected patient population is 15%, using this method, only 15% of patients will continue on therapy unnecessarily until week 8, while using traditional imaging methods, 85% of patients will continue on treatment unnecessarily until week 8. This example assumes there are 100 patients and 15 will be true responders. Yet the present method will detect 30 patients to continue on study. The other 70 patients will move off trial after a day 5 assessment. Of the 30 patients who continue on study, 15 will respond and 15 will be unnecessarily treated until week 8+. But 15 of 100 unnecessarily treated is a lot better than 85 of 100. Further, a 50% response rate in oncology is considered a breakthrough therapy and no randomized trial is needed. The trade-off is that everyone will get a single dose of drug, but if the drug is relatively nontoxic (such as a targeted therapy) that should be acceptable. Accordingly, the present disclosed method is a non-traditional paradigm that will minimize side effect exposure when treatment benefit is not being well realized. This will also help to better design clinical drug trials such that better measurement criteria of treatment effectiveness can be utilized to better measure key risk/benefit criteria.

One technique that can be used for additional information is using FDG Positron Emission Tomography (PET) for treatment monitoring. An FDG PET (Positron Emission Tomography) scan is a radiological test that looks at tissue functioning, such as metabolic rates of tissues. With an FDG PET scan, a small amount of a radioactive sugar is injected into the blood. Cells that are growing, such as tumor cells, use sugar, and can be seen on 3-dimensional imaging. However, treatment monitoring, often comes at a later time during the course of treatment, after a patient has been exposed to multiple side effects of a treatment that may or may not have been effective. Efforts to determine particular drug effectiveness have been looking at personalized pharmacokinetic profiles. For example, uses of PET imaging has been done in combination with radiolabeled TM's (tyrosine kinase inhibitors such as erlotnib and gefitinib) to measure pharmacokinetics of the tyrosine kinase inhibitors. However, the dose of the radiolabeled TM's has been a microdose, not a full therapeutic dose. Pharmacokinetic analysis has been used to stratify patients. It was hypothesized that a patient's response to TKIs are dependent on achieving pharmacological active drug levels in tumor tissue and quantitative PET imaging can predict kinase inhibitor tumor concentrations. In this regard, VU Medical Center in the Netherlands is running a clinical trial to determine whether tumor concentrations of TKIs at pharmacological active doses can be predicted from PET studies using tracer amounts (micro-doses) of radiolabeled kinase inhibitors. However, the use of PET scans in this regard requires a correlation between local tumor drug concentration and specific efficacy.

As an additional measuring process, in addition to PET scans and run at the same time as the PET scans, a quantitative amount of circulating tumor DNA (ctDNA) in blood (plasma), urine, and other bodily fluids can be measured. What is measured is a relevant (to the tumor) biomarkers, which are specific oncogenes from cancer patients that have been shown to be correlated with radiographic (e.g., CAT, PET, MRI) indication of response to therapy. ctDNA or cell-free tumor DNA present in a cancer patient's blood (plasma) and subsequent urine that is derived from and the consequence of tumor cell death (apoptosis or necrosis) or shed in other ways. The relative amounts of ctDNA correlates with the tumor burden (i.e., number of tumor cells) within a cancer patient with higher amounts of ctDNA associated with greater tumor burden. The amount of ctDNA within a cancer patient's plasma and urine can be measured by quantitating the number of DNA copies present for specific oncogene aberrations derived from tumor cells. Therefore, as tumor burden increases in a patient, there is greater absolute number of tumor cell turnover (i.e., cell death) or other processes that shed tumor DNA, which equates to a larger number of copies of oncogenic tumor DNA present within plasma and subsequently urine. The relative measurement of ctDNA can be used to determine effectiveness of a cancer therapy and has been demonstrated for numerous metastatic cancers including breast, lung and colorectal cancer. For example, levels of the oncogene KRAS within metastatic cancer patients is correlated with responsiveness to chemotherapy. In histiocytic disorders, a cancer-related disease, approximately 50% of patients harbor a BRAF oncogene (i.e., BRAFV600E) and these patients responded to BRAF inhibitors. Relative measurement of BRAFV600E in urine from these patients receiving a BRAF inhibitor decreased at least 5-fold from baseline (prior to therapy) as compared to post-therapy and correlated with radiographic response. Furthermore, these changes in ctDNA levels are observed within days.

Comparative measurement of ctDNA levels from a single therapeutic dose at baseline and within days (1-14) post-therapy (and the serial pattern examined) is used as a responsive biomarker for determining which patients will respond to continued therapy. For example, for Ewing sarcoma patients, monitoring ctDNA levels of hybrid genes of 5′EWSR1 fused to parts of either FL11, ERG of EVT1. For instance, in lung cancer, this technique shows that an initial rise in ctDNA and then fall within days is predictive or response. In Marchetti et al. (J. Thoracic Oncl. 10:1437-1443, October 2015, and not prior art to the priority provisional application) an early prediction of a response to tyrosine kinase inhibitors by quantifying EGFR mutations (a biomarker) was found that a PCR test and sequencing of plasma found for EGFR a progressive decrease in SQ1 decrease starting from day 4 after therapy.

In conducting a biomarker analysis, the sequence of the point mutation or deletion mutation is detected with tissue (solid tumor) or plasma, urine or other body fluid samples collected before and at multiple times after (but within 14 days) the initiation of treatment. Preferably, the after samples are collected before a second dose or a second round of treatment is initiated in order to determine the effectiveness of only the single first dose or round of treatment. The initial samples (before treatment are analyzed with PCR primer sets bracketed around the cancer marker location to determine and confirm an appropriate tumor marker. For example, for EGFR mutation analysis with 42 patients, there was an exon 19 deletion in 31 cases (74%) and an L858R point mutation in exon 21 in 11 cases (26%).

Using early response to predict true response is done by comparing PET FDG at days 1 to 14, with baseline (pretherapy) PET-FDG, or comparing ctDNA levels of the biomarker on days 1 to 14, with baseline (pretherapy) ct DNA (in blood, urine or other bodily fluids) or integrating both parameters (PET-FDG and ct DNA changes). Other forms of PET scans, such as PET-FLT, may also be used.

Other molecular markers that could be used, but will be specific to the tumor to be treated, are selected from Table 1A and 1B.

TABLE 1A
INTEGRATED GENE LIST (includes mutations, amplifications, deletions and other changes)

| | | | | | | | |
|---|---|---|---|---|---|---|---|
| ABCB1 | ARFRP1 | AURKC | BIRC3 | C11orf30 | CD274 | CD36 | CDK2 |
| | | | | (EMSY) | (PDL1) | | |
| ABCC1 | ARID1A | AVPR2 | BIRC5 | C17orf39 | CDX2 | CD37 | CDK4 |
| | | | | (GID4) | | | |
| ABCC2 | ARID1B | AXIN1 | BLM | CA9 | CHIC2 | CD38 | CDK6 |
| ABCC6 | ARID2 | AXL | BMI1 | CAD | CHN1 | CD4 | CDK7 |
| ABCG2 | ASMTL | AR | BCL11A | CALCA | CLP1 | CD40 | CDK8 |
| ABL1 | ACSL6 | ARAF | BCL3 | CALCR | CLTC | CD44 | CDK9 |
| ABL2 | AFF1 | B2M | BCL9 | CALM1 | CLTCL1 | CD52 | CDKN1A |
| ACE | AFF4 | BAD | BMP10 | CALM2 | CNTRL | CD58 | CDKN1B |
| | | | | | (CEP110) | | |
| ACPP | ARHGAP26 | BAP1 | BRAF | CALM3 | COL1A1 | CD70 | CDKN2A |
| | (GRAF) | | | | | | |
| ACTB | ARHGEF12 | BARD1 | BRCA1 | CARD11 | CREB3L1 | CD74 | CDKN2B |
| ACVR1B | ARNT | BBC3 | BRCA2 | CASP3 | CREB3L2 | CD79A | CDKN2C |
| ACVRL1 | ATF1 | BCL10 | BRD4 | CASP7 | CSF1 | CD79B | CDKN2D |
| ADA | ATG5 | BCL11B | BRIP1 | CASP8 | CCNG1 | CDA | CEACAM5 |
| ADAM15 | ATIC | BCL2 | BRSK1 | CASP9 | CCR4 | CDC7 | CEBPA |
| AFP | ASXL1 | BCL2A1 | BTG1 | CBFB | CCR5 | CDC73 | CENPE |
| AKT1 | ATM | BCL2L1 | BTG2 | CBL | CCT6B | CDH1 | CHD2 |
| AKT2 | ATP1A3 | BCL2L2 | BTK | CBR3 | CD109 | CDH2 | CHD4 |
| AKT3 | ATP7A | BCL6 | BTLA | CCL3 | CD151 | CDH20 | CHEK1 |
| ALK | ATR | BCL7A | BUB1 | CCND1 | CD19 | CDH5 | CHEK2 |
| ALOX12 | ATRX | BCOR | CAMTA1 | CCND2 | CD22 | CDK1 | CIAPIN1 |
| ALOX12B | AURKA | BCORL1 | CARS | CCND3 | CD248 | CDK12 | CIC |
| AMER1 | AURKB | BCR | CBFA2T3 | CCNE1 | CPS1 | CRLF2 | CSF3R |
| (FAM123B | | | | | | | |
| or WTX) | | | | | | | |
| ANGPT1 | APC | BGLAP | CIITA | CLDN18 | CREBBP | CSF1R | CTCF |
| ANGPT2 | APH1A | BIRC2 | CKS1B | CLU | CRKL | CSF2 | CTLA4 |
| CTNNA1 | DHFR | EP300 | FOXO4 | FGFR2 | GATA1 | HOXD13 | HOXD13 |
| CTNNB1 | DIABLO | EPCAM | FSTL3 | FGFR3 | GATA2 | HBB | HSPA5 |
| CTSG | DICER1 | EPHA3 | FUS | FGFR4 | GATA3 | HBEGF | HLF |
| CUL3 | DKK1 | EPHA5 | F13B | FH | GATA4 | HDAC1 | HMGA1 |
| CUX1 | DLL4 | EPHA6 | F2 | FHIT | GATA6 | HDAC11 | HMGA2 |
| CXCL12 | DNM2 | EPHA7 | F3 | FLCN | GDF2 | HDAC2 | HOXA11 |
| CXCR1 | DNMT3A | EPHB1 | F5 | FLT1 | GGH | HDAC4 | HOXA13 |
| CXCR2 | DOT1L | EPHB4 | FAF1 | FLT3 | GLI1 | HDAC6 | HOXA9 |
| CXCR4 | DPP4 | EPHB6 | FAM46C | FLT3LG | GLP2R | HDAC7 | HOXC11 |
| CYBA | DPYD | ERBB2 | FANCA | FLT4 | GNA11 | HFE | HOXC13 |
| CYLD | DTX1 | ERBB3 | FANCC | FLYWCH1 | GNA12 | HGF | HOXD11 |
| CYP11B1 | DUSP2 | ERBB4 | FANCD2 | FOLH1 | GNA13 | HIF1A | IL11RA |
| CYP11B2 | DUSP9 | ERCC2 | FANCE | FOLR1 | GNAQ | HIST1H1C | IGH |
| CYP17A1 | EIF4A2 | ERCC5 | FANCF | FOXL2 | GNAS | HIST1H1D | IGK |
| CYP19A1 | ELF4 | ERG | FANCG | FOXO1 | GNRHR | HIST1H1E | IGL |
| CYP1B1 | ELL | ERRFI1 | FANCL | FOXO3 | GPC3 | HIST1H2AC | IL3 |
| CYP2C19 | ELN | ESR1 | FAS | FOXP1 | GPR124 | HIST1H2AG | IFNA2 |
| | | | (TNFRSF6) | | | | |
| CYP2C8 | EPOR | ESR2 | FASLG | FOXP4 | GRIN2A | HIST1H2AL | IFNB1 |
| CYP2C9 | EPS15 | ETS1 | FAT1 | FRS2 | GRIN3B | HIST1H2AM | IFNG |
| CYP2D6 | E2F1 | ETV1 | FBXO11 | FUBP1 | GRM3 | HIST1H2BC | IGF1R |
| CYP3A4 | EBF1 | ETV4 | FBXO31 | FYN | GSK3B | HIST1H2BJ | IGF2 |
| CYP4B1 | ECT2L | ETV5 | FBXW7 | FZD1 | GSTO1 | HIST1H2BK | IGF2R |
| DDIT3 | EDNRA | ETV6 | FGF1 | FZD10 | GSTO2 | HIST1H2BO | IKBKE |
| DDX10 | EDNRB | EWSR1 | FGF10 | FZD2 | GSTP1 | HIST1H3B | IKZF1 |
| DDX6 | EED | EXOSC6 | FGF14 | FZD5 | GTSE1 | HNF1A | IKZF2 |
| DEK | EEF2 | EZH2 | FGF19 | FZD7 | GUCY1A2 | HOXA3 | IKZF3 |
| DUSP22 | EGFL7 | FCGR2B | FGF2 | FZD8 | G6PD | HPSE | IL13RA2 |
| DAXX | EGFR | FCRL4 | FGF23 | GAS7 | HERPUD1 | HRAS | IL1A |
| DCT | EIF4EBP1 | FEV | FGF3 | GMPS | HEY1 | HRH2 | IL2 |
| DDR2 | ELP2 | FGFR1OP | FGF4 | GPHN | HIP1 | HSD3B1 | IL21R |
| DDX3X | EML4 | FLI1 | FGF6 | GABRA6 | HIST1H4I | HSP90AA1 | IL25 |
| DDX5 | ENG | FNBP1 | FGFR1 | GADD45B | HSP90AB1 | HSP90B1 | IL29 |
| ICK | JUN | LRP1B | MED12 | MLLT3 | NRAS | PAK3 | PLA2G10 |
| ID3 | KAT6A | LRP2 | MEF2B | MLLT4 | NRP2 | PALB2 | PLA2G12A |
| | (MYST3) | | | (AF6) | | | |
| IDH1 | KDSR | LRP6 | MEF2C | MLLT6 | NSD1 | PARK2 | PLA2G12B |
| IDH2 | KIF5B | LRRK2 | MEFV | MN1 | NT5C2 | PARP1 | PLA2G1B |
| IL2RA | KMT2A | LTA | MEN1 | MNX1 | NTRK1 | PARP2 | PLA2G2A |
| | (MLL) | | | | | | |
| IL4 | KDM2B | LTF | MET | MSI2 | NTRK2 | PARP8 | PLA2G2D |
| IL4R | KDM4C | LTK | MGMT | MSN | NTRK3 | PASK | PLA2G2E |
| IL7R | KDM5A | LYN | MIB1 | MUC1 | NUP93 | PAX5 | PLA2G2F |
| INHBA | KDM5C | LZTR1 | MITF | MYB | NUP98 | PBRM1 | PLA2G3 |
| INPP4B | KDM6A | LMO2 | MKI67 | MYH9 | NACA | PC | PLA2G5 |
| INPP5D | KDR | MAF | MLH1 | MYO18A | NBEAP1 | PCBP1 | PLA2G6 |
| (SHIP) | | | | | (BCL8) | | |
| INSR | KEAP1 | MAFB | MMP2 | NAE1 | NCOA2 | PCLO | PLAU |
| IRF1 | KEL | MAGEA1 | MPL | NAMPT | NDRG1 | PDCD1 | PLCG1 |
| IRF2 | KIF11 | MAGEA4 | MRE11A | NAT2 | NFKB2 | PDCD11 | PLCG2 |
| IRF4 | KIT | MAGED1 | MS4A1 | NCF2 | NIN | PDCD1LG2 | PLK1 |
| | | | | | | (PDL2) | |
| IRF8 | KLF4 | MAGI2 | MSH2 | NCL | NUMA1 | PDGFB | PLK4 |
| IRS2 | KLHL6 | MALT1 | MSH3 | NCOR2 | NUP214 | PDGFRA | PMP22 |
| ITGA1 | KLK2 | MAP2K1 | MSH6 | NCSTN | NUTM2A | PDGFRB | PMS2 |
| ITGA5 | KLK3 | MAP2K2 | MST1R | NF1 | NR4A3 | PDK1 | PNP |
| ITGAM | KMT2C | MAP2K4 | MSTN | NF2 | OMD | PDPK1 | POLD1 |
| | (MLL3) | | | | | | |
| ITGAV | KMT2D | MAP3K1 | MTF1 | NFE2L2 | OPRD1 | PGF | POLE |
| | (MLL2) | | | | | | |
| ITGB1 | KRAS | MAP3K14 | MTHFR | NFKB1 | PAX3 | PGR | POT1 |
| ITGB2 | LASP1 | MAP3K6 | MTOR | NFKBIA | PAX7 | PHF6 | PPARA |
| ITGB3 | LCP1 | MAP3K7 | MTR | NGF | PBX1 | PHLPP2 | PPARD |
| ITGB5 | LPP | MAPK1 | MUTYH | NKX2-1 | PCM1 | PIK3C2B | PPARG |
| ITGB6 | LYL1 | MAPK3 | MYC | NOD1 | PCSK7 | PIK3CA | PPP2R1A |
| ITK | LAG3 | MCL1 | MYCL | NOD2 | PDE4DIP | PIK3CB | PRAME |
| | | | (MYCL1) | | | | |
| ITPA | LEF1 | MDM2 | MYCN | NOTCH1 | PER1 | PIK3CD | PRDM1 |
| JAZF1 | LGALS1 | MDM4 | MYD88 | NOTCH2 | PHF1 | PIK3CG | PREX2 |
| JAK1 | LHCGR | MED1 | MYH11 | NOTCH3 | PICALM | PIK3R1 | PRKAR1A |
| JAK2 | LMO1 | MDS2 | MLF1 | NPM1 | P2RX7 | PIK3R2 | PRKCA |
| JAK3 | LOXL2 | MECOM | MLLT1 | NQO1 | P2RY8 | PGR | PRKCB |
| | | | (ENL) | | | | |
| JARID2 | LPA | MKL1 | MLLT10 | NR4A1 | PAG1 | PIM1 | PAFAH1B2 |
| | | | (AF10) | | | | |
| PLAG1 | RAD51 | S100A9 | SSX2 | SSTR2 | TPM4 | TNFRSF14 | UBB |
| PML | RAD5IL3 | S1PR1 | SSX4 | SSTR3 | TRIM24 | TNFRSF17 | UBC |
| POU2AF1 | RAF1 | S1PR2 | STL | SSTR4 | TRIP11 | TNFRSF4 | UGT1A1 |
| PPP1CB | RANBP2 | SDHA | SLC7A11 | SSTR5 | TTL | TNFRSF8 | UGT1A7 |
| PRDM16 | RARA | SDHB | SLCO1B1 | STAT3 | TEC | TNFRSF9 | UMPS |
| PRRX1 | RASGEF1A | SDHC | SLIT2 | STAT4 | TEK | TNFSF10 | USP9X |
| PSIP1 | RB1 | SDHD | SMAD2 | STAT5A | TERC | TNFSF13 | VCAM1 |
| PRKCI | RBM10 | SELL | SMAD3 | STAT5B | TERT | TNFSF13B | VDR |
| PRKDC | RELN | SELP | SMAD4 | STAT6 | TET1 | TNKS | VEGFA |
| PRLR | RET | SERP2 | SMARCA1 | STK11 | TET2 | TOP1 | VEGFB |
| PRSS8 | RHEB | SERPINA1 | SMARCA4 | SUFU | TGFB1 | TOP2A | VEGFC |
| PSMB5 | RHOA | SERPINE1 | SMARCB1 | SULTIC4 | TGFBR1 | TOR1A | VHL |
| PSMB8 | RICTOR | SETBP1 | SMC1A | SUZ12 | TGFBR2 | TP53 | VKORC1 |
| PTCH1 | RNF43 | SETD2 | SMC3 | SYK | TGM2 | TP63 | VPS4B |
| PTCH2 | ROCK2 | SF3B1 | SMC4 | T | TH | TP73 | VWF |
| PTEN | ROS1 | SFTPC | SMO | TACR1 | TLL2 | TPMT | WHSC1 |
| | | | | | | | (MMSET |
| | | | | | | | or |
| | | | | | | | NSD2) |
| PTGS2 | RPE65 | SGK1 | SNCAIP | TAF1 | TLR2 | TRAF2 | WHSCIL1 |
| PTH | RPS27A | SHH | SOCS1 | TBL1XR1 | TLR3 | TRAF3 | WDR90 |
| PTK2B | RPTOR | SLC10A3 | SOCS2 | TBX22 | TLR4 | TRAF5 | WISP3 |
| PTPN11 | RRM2 | SLC16A1 | SOCS3 | TBX3 | TLR5 | TRPM8 | WT1 |
| PTPN2 | RUNX1 | SLC19A1 | SOD2 | TAL1 | TLR7 | TSC1 | XBP1 |
| PTPN6 | RUNX3 | SLC29A1 | SOX10 | TAL2 | TLR8 | TSC2 | XIAP |
| (SHP-1) | | | | | | | |
| PTPRC | RABEP1 | SLC5A5 | SOX2 | TAF15 | TLR9 | TSHR | XPC |
| PTPRD | RALGDS | SLC6A2 | SOX9 | TCF3 | TMEM30A | TUSC2 | XPO1 |
| | | | | (E2A) | | | |
| PTPRO | RAP1GDS1 | SEC31A | SPEN | TCL1A | TMPRSS2 | TUSC3 | XRCC1 |
| | | | | (TCL1) | | | |
| PRRX1 | RBM15 | SET | SPG7 | TFE3 | TMSB4XP8 | TYK2 | XRCC2 |
| | | | | | (TMSL3) | | |
| PSIP1 | RHOH | SH3GL1 | SPOP | TFG | TNC | TYMS | YPEL5 |
| PTK7 | RNF213 | SLC1A2 | SPP1 | TFPT | TNF | TYR | YES1 |
| QKI | RPL22 | SNX29 | SPTA1 | TFRC | TNFAIP3 | USP6 | YY1AP1 |
| | | (RUNDC2A) | | | | | |
| RAC1 | RPN1 | SRSF3 | SRC | TLX1 | TNFRSF10A | U2AF1 | ZBTB2 |
| RAD21 | RUNX1T1 | SS18 | SRSF2 | TLX3 | TNFRSF10B | U2AF2 | ZEB2 |
| | (ETO) | | | | | | |
| RAD50 | RUNX2 | SSX1 | SSTR1 | TPM3 | TNFRSF11A | UBA52 | ZMYM3 |
| ZBTB16 | | | | | | | |
| ZMYM2 | | | | | | | |
| ZNF384 | | | | | | | |
| ZNF521 | | | | | | | |
| ZNF217 | | | | | | | |
| ZNF24 | | | | | | | |
| (ZSCAN3) | | | | | | | |
| ZNF703 | | | | | | | |
| ZRSR2 | | | | | | | |

TABLE 1B
Rearrangements and other Alterations

| | | |
|---|---|---|
| ALK | FGFR1 | |
| ALK | FGFR1 | PDGFRA |
| BCL2 | FGFR2 | PDGFRB |
| BCL6 | FGFR3 | RAF1 |
| BCR | IGH | RARA |
| BRAF | IGK | RET |
| BRCA1 | IGL | ROS1 |
| BRCA2 | JAK1 | SYT/SSX1 SYT/SSX2 |
| BRD4 | JAK2 | TMPRSS2 |
| CCND1 | KIT | TRG |
| CRLF2 | KMT2A (MLL) | |
| EGFR | MSH2 | |
| EPOR | MYB | |
| ETV1 | MYC | |
| ETV4 | NOTCH2 | |
| ETV5 | NTRK1 | |
| ETV6 | NTRK2 | |
| EWSR1 | PAX3/FKHR | |
| | PAX7/FKHR | |
| EWS-FLI | | |
| EWS/FLI1, type 1 | | |
| EWS/FLI1, type 2 | | |
| EWS/ERG | | |
| EWS/ETV1 | | |
| EWS/ETV4 | | |
| EWS/FEV | | |

We claim:

1. A process for determining whether a patient would benefit from cancer treatment with a particular therapeutic, comprising (a) conducting a baseline PET scan in a patient by determining tumor tissue metabolic rate, (b) providing a single potentially effective dose of a therapeutic to the patient, (c) conducting a second PET scan of the patient by determining tumor tissue metabolic rate, wherein the second PET scan is conducted 1-14 days following the dose of the therapeutic, and (d) comparing the results of the first PET scan to the second PET scan to determine a response in the PET scan results such that at least a 1% reduction in tumor tissue metabolic rate indicates that the patient would benefit to treat the tumor with the therapeutic.

2. The process for determining whether a patient would benefit for cancer treatment with a particular therapeutic of claim 1, wherein the PET scans determines lesion volume VOI ROIs to obtain maximum SUVmax and SUV SUV minimum SUVmin.

3. The process for determining whether a patient would benefit from cancer treatment with a particular therapeutic of claim 1, further comprising conducting a DNA aberration marker test to quantitate a plasma-free DNA alteration of a cancer marker selected from the list of markers in Tables 1A and 1B.

4. The process for determining whether a patient would benefit from cancer treatment with a particular therapeutic of claim 3, wherein the DNA aberration marker test conducted at substantially the same time as the first PET scan and the second PET scan.

6. The process for determining whether a patient would benefit for cancer treatment with a particular therapeutic of claim 1, wherein the second PET scan is conducted within 14 days after completion of a first dose of a therapeutic to the patient.

7. The process for determining whether a patient would benefit for cancer treatment with a particular therapeutic of claim 6, wherein the second PET scan is conducted form 1-10 days after completion of a first dose of a therapeutic to the patient.

8. A process for determining patient subgroup inclusion in a clinical trial of a targeted therapy for cancer, comprising (a) conducting a baseline PET FDG or PET FLT scan of the tumor region in a patient by determining baseline tumor tissue metabolic rate, (b) providing a single dose of a clinical trial test targeted therapeutic to the patient, (c) conducting a second PET FDG or PET FLT scan within 1-14 days following the single dose, by determining tumor tissue metabolic rate, and (d) comparing the results of the first PET scan to the second PET scan to determine an imaging response in the PET scan results, whereby at least a 1% reduction in tumor tissue metabolic rate indicates that the patient would be included in the clinical trial subgroup.

9. The process for determining patient subgroup inclusion in a clinical trial of a targeted therapy for cancer of claim 8, further comprising conducting a parallel before and after measurement or serial measurements of the level of circulating tumor DNA (ctDNA) for specific oncogene mutations/alterations from a response biomarker specific for a tumor to identify early patient response to drug therapy, wherein the response biomarker is selected from the group consisting of the response biomarkers in Tables 1A and 1B.

10. A process for determining whether a patient would benefit for cancer treatment with a particular therapeutic, comprising (a) conducting a baseline measurement of the level of circulating tumor DNA (ctDNA) for specific oncogene mutations/alterations from a response biomarker specific for a tumor to identify early patient response to drug therapy, wherein the response biomarker is selected from the group consisting of the response biomarkers in Tables 1A and 1B, (b) providing a single potentially effective dose of a therapeutic to the patient, (c) conducting a second measurement of the level of circulating tumor DNA (ctDNA) for specific oncogene mutations/alterations from a response biomarker specific for a tumor, wherein the response biomarker is selected from the group consisting of the response biomarkers in Tables 1A and 1B, wherein the second or serial measurement(s) are conducted 1-14 days following the dose of the therapeutic, and (d) comparing the results of the first measurement to the second measurement to determine if at least a 1% reduction indicates that the patient would benefit to continue to treat the tumor with the therapeutic.

11. The process for determining whether a patient would benefit for cancer treatment with a particular therapeutic of claim 10, wherein the second or serial measurement(s) are conducted from 1-10 days after completion of a first dose of a therapeutic to the patient.
